(19) United States
(12) Patent Application Publication
(10) Pub. No.: US 2007/0078408 A1
(43) Pub. Date: Apr. 5, 2007

(54) Disposable safety syringe for dispensing anesthetic

(76) Inventor: Hsien Tsung Wang, Taipei (TW)

Correspondence Address: BRUCE H. TROXELL, SUITE 1404, 5205 LEESBURG PIKE, FALLS CHURCH, VA, 22041, US

(21) Appl. No.: 11/211,609

(22) Filed: Aug. 26, 2005

(51) Int. Cl. A61M 5/32; A61M 5/00

(52) U.S. Cl. 604/198; 604/110

(57) ABSTRACT

A disposable syringe comprises an arm, a plunger for receiving a fluid cartridge and including a surface projection, a needle assembly secured to a forward nose of the plunger, and a syringe barrel including a lengthwise slot on its surface with the projection disposed therein, a locking trough proximate a curved rear end of the slot, and a flexible latch provided between the rear end of the slot and the locking trough. After dispensing anesthetic, pulling the plunger rearward will retract the needle assembly into the syringe barrel until the projection moves from a forward end of the slot to the locking trough by passing the latch for locking the projection in the locking trough.

### BACKGROUND OF THE INVENTION

1. Field of Invention

The present invention relates to syringes and more particularly to a disposable syringe for dispensing anesthetic with safety arrangement for concealing and locking a needle after use.

2. Description of Related Art

Conventionally, a syringe for dispensing anesthetic is made of stainless steel and which is relatively high in cost. Thus, it is typical of sterilizing the used syringe rather than discarding it. However, in often times microorganisms may be not completely killed in the sterilization. As to the sterilization, it involves steps of removing a needle from a syringe and sterilizing the syringe thereafter. However, it is highly possible that the needle may accidentally prick fingers of a medical worker and thereby contaminate the medical worker with microorganisms. However, so far as the present inventor is aware, there is no such syringe available on the market that completely and satisfactorily solves the safety problem. Thus, a need for improvement exists.

### SUMMARY OF THE INVENTION

It is therefore an object of the present invention to provide a safety syringe for dispensing anesthetic in which the syringe is formed of a plastic material and the syringe is disposable after its use (i.e., no sterilization required) and to thereby eliminate the possibility of contaminating a medical worker with microorganisms due to incomplete sterilization.

It is another object of the present invention to provide a safety syringe for dispensing anesthetic in which a plunger having a needle attached to its forward nose is adapted to retract into a hollow syringe barrel and is locked therein after its use, then the syringe is discarded, and to thereby eliminate the possibility of contaminating a medical worker with microorganisms due to accidental pricking and to prevent the used syringe from being used again after sterilization.

To achieve the above and other objects, the present invention provides a retractable syringe for dispensing anesthetic comprising a fluid cartridge including a rear sliding member; an elongated arm including a rear ring for enabling a finger to insert into, and a forward disk having a diameter slightly smaller than that of the sliding member; a hollow, cylindrical plunger for receiving the fluid cartridge and including a projection on its outer surface proximate a forward end, outer threads at a rear end, and a forward threaded nose; a wing nut including a forward hollow enlargement having inner threads threadedly secured to the outer threads of the plunger for fastening the wing nut and the plunger together, and an axial channel such that the arm is adapted to insert through the channel and the enlargement into the plunger to engage the sliding member; a needle assembly including a housing having inner threads threadedly secured to the nose, a needle extended forward from the housing, and a slender metal tube extended rearward from a rear end of the needle into the fluid cartridge for forming a fluid path from the fluid cartridge to the needle via the metal tube; and an elongated syringe barrel including a lengthwise slot on its surface with the projection disposed in the slot, a locking trough proximate a curved rear end of the slot, and a flexible latch provided between the rear end of the slot and the locking trough; whereby in an anesthetic dispensing operation pushing the arm forward will project the needle from the syringe barrel and move the projection to a forward end of the slot, and after the dispensing operation pulling the plunger rearward will retract the needle assembly into the syringe barrel until the projection moves from the forward end of the slot to the locking trough by passing the rear end of the slot and the latch for locking the projection in the locking trough.

The above and other objects, features and advantages of the present invention will become apparent from the following detailed description taken with the accompanying drawings.

### BRIEF DESCRIPTION OF THE DRAWINGS

FIG. 1 is a perspective view of a preferred embodiment of syringe for dispensing anesthetic according to the invention;

FIG. 2 is an exploded perspective view of the syringe of FIG. 1;

FIGS. 3 and 4 are side views showing the syringe of FIG. 1 in a ready use state for fluid dispensing operation and in an after use state respectively in which in the after use state a plunger having a needle attached to its forward nose is retracted into a hollow syringe barrel and is locked therein;

FIG. 4A is a partial enlarged view of FIG. 4; and

FIG. 5 is a cross-sectional view of FIG. 3.

### DETAILED DESCRIPTION OF THE INVENTION

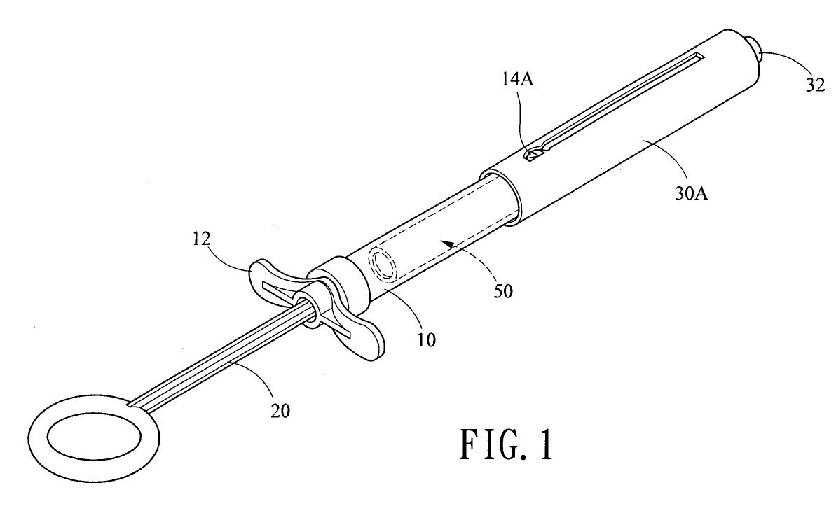

Referring to FIGS. 1 to 5, a disposable safety syringe for dispensing anesthetic in accordance with a preferred embodiment of the invention comprises a plunger 10, an arm 20, a syringe barrel 30A, a hypodermic needle assembly 40, and a fluid cartridge 50. Each component is discussed in detailed below.

The fluid cartridge 50 is a cylindrical container made of glass and is commercially available. The fluid cartridge 50 comprises a forward extension 51 formed of flexible material, and a cylindrical sliding member 52 provided in a rear end in a fully charged state of the fluid cartridge 50 and adapted to slide toward the extension 51 for extracting fluid from a space defined by the extension 51 and the sliding member 52 in a fluid dispensing operation.

The hollow, cylindrical plunger 10 is formed of a plastic material and comprises an internal space 11 for receiving the fluid cartridge 50, a rear end 13 having outer threads 130, a forward nose 15 having a diameter smaller than that of the main body of the plunger 10 and outer threads, and a projection 14A on its outer surface proximate the nose 15 in which the projection 14A has a section of triangle having a higher forward side (i.e., a top surface of the projection 14A is slanted from the side facing the nose 15 to a rear vertex).

The plunger 10 further comprises a separate wing nut 12 including an axial bore 120 and a forward hollow, cylindrical enlargement 121 having inner threads 122 threadedly secured to the outer threads 130 for fastening the wing nut 12 and the plunger 10 together.

The arm 20 is elongated and comprises a rear ring 22 as a handle for enabling a finger to insert into, a forward disk 21 having a diameter slightly smaller than that of the sliding member 52, and a separate rubber ring 23. The arm 20 is adapted to insert through the bore 120, the rubber ring 23, the enlargement 121, and a rear portion of the space 11 to urge against the sliding member 52 in which the rubber ring 23 is disposed in close proximity to rear of the disk 21 so as to prevent the arm 20 from disengaging with the wing nut 12 in pushing or pulling operation of the arm 20 by holding the ring 22 while dispensing anesthetic.

The needle assembly 40 comprises a rear conic housing having inner threads 403 threadedly secured to the threaded nose 15 for fastening the needle assembly 40 and the plunger 10 together, a needle 402 extended forward from the housing, and a slender metal tube 401 extended rearward from a rear end of the needle 402 in the housing through the extension 51 into the fluid cartridge 50 such that fluid communication from the fluid cartridge 50 to the needle 402 via the metal tube 401 can be effected.

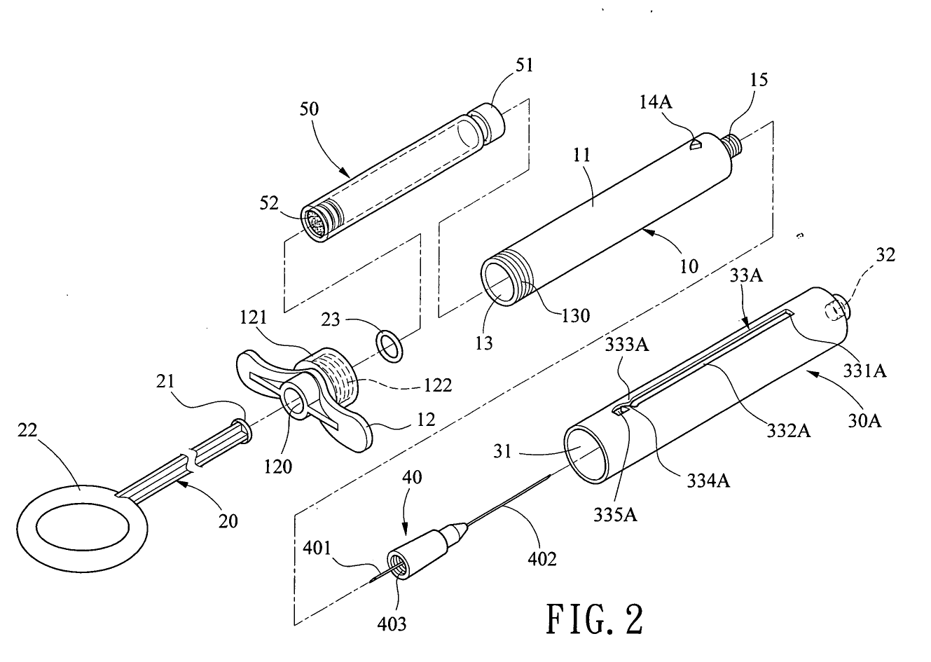

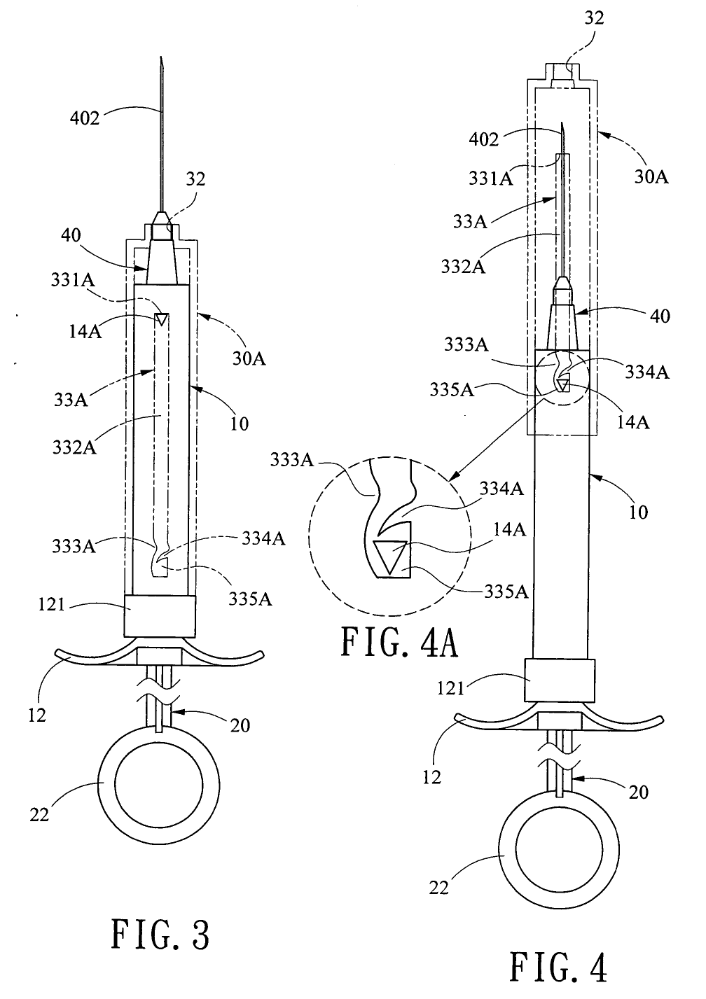

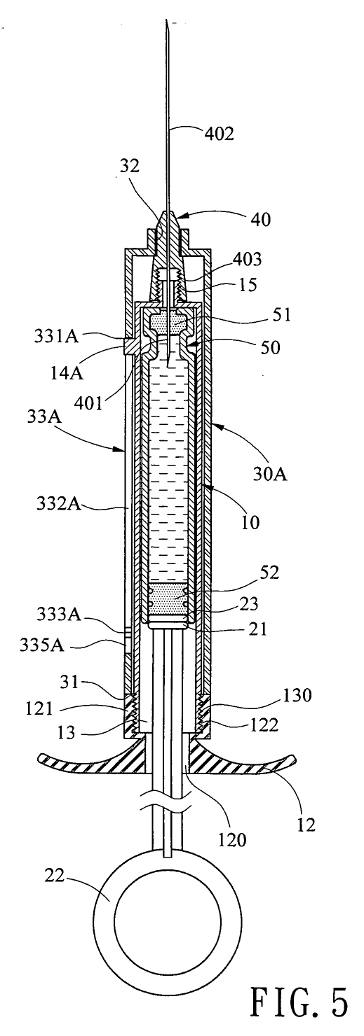

The syringe barrel 30A is an elongated, cylindrical member made of a plastic material and comprises an axial bore 31 having a diameter slightly larger than an outer diameter of the plunger 10 such that the assembled needle assembly 40 and the plunger 10 can be disposed in the bore 31 (see FIGS. 2 and 5). The syringe barrel 30A further comprises a lengthwise slot 332A on its surface with the projection 14A movably disposed in the slot 332A after assembly and stopped at a forward end 331A thereof in a ready use state for fluid dispensing operation (see FIGS. 3 and 5), a locking trough 335A proximate a curved rear end 333A of the slot 332A, and a flexible latch 334A provided between the rear end 333A and the locking trough 335A. In accordance with the preferred embodiment of the present invention, the flexible latch 334A, as shown in FIGS. 4 and 4A, as its forming orientation is toward rear end of the slot 332A. So, the flexible latch 334A will be deformed when the projection 14A is passed through from the forward end 331A of the slot 332A to the rear end 333A to enable the projection 14A to further enter into the locking trough 335A. However, the flexible latch 334A will not be deformed when the projection 14A moves backward from the locking trough 335A to the rear end 333A, so as soon as the projection 14A locked in the locking trough 335A will no longer have the projection 14A moved out any more. In a fluid dispensing operation, a medical worker may use one hand to hold the syringe barrel 30A and use the other hand to push the arm 20 forward for projecting the needle 402 from a forward opening 32 of the syringe barrel 30A prior to inserting into the body of a patient. After dispensing fluid (e.g., anesthetic), a medical worker can pull the plunger 10 rearward (i.e., the syringe barrel 30A moves forward) to retract the needle assembly 40 into the syringe barrel 30A until the projection 14A moves from the forward end 331A to the locking trough 335A by passing the rear end 333A and the latch 334A as mentioned above. As shown in FIG. 4, the projection 14A is therefore locked in the locking trough 335A (i.e., the needle assembly 40 thus locked in the syringe barrel 30A). The whole syringe is no longer useful and thereby eliminates the possibility of contaminating a medical worker with microorganisms due to accidental pricking and prevents the used syringe from being used again after sterilization.

Note that the plunger 10, the arm 20, the housing of the needle assembly 40, and the syringe barrel 30A are all made of a plastic material. Thus, the whole syringe can be discarded after use since the plunger 10 and the needle assembly 40 are concealed and locked by the syringe barrel 30A. Also, the discarded syringe is adapted to properly dispose without causing environmental pollution. Further, in other embodiments the projection 14A may have a shape other than one having a section of triangle in this embodiment.

While the invention herein disclosed has been described by means of specific embodiments, numerous modifications and variations could be made thereto by those skilled in the art without departing from the scope and spirit of the invention set forth in the claims.

What is claimed is:

1. A retractable syringe for dispensing anesthetic comprising:
a fluid cartridge including a rear sliding member;
an elongated arm including a rear ring for enabling a finger to insert into, and a forward disk having a diameter slightly smaller than that of the sliding member;
a hollow, cylindrical plunger for receiving the fluid cartridge and including a projection on its outer surface proximate a forward end, a plurality of outer threads at a rear end, and a forward threaded nose;
a wing nut including a forward hollow enlargement having a plurality of inner threads threadedly secured to the outer threads of the plunger for fastening the wing nut and the plunger together, and an axial rear channel such that the arm is adapted to insert through the rear channel and the enlargement into the plunger to engage the sliding member;
a needle assembly including a housing having inner threads threadedly secured to the nose, a needle extended forward from the housing, and a slender metal tube extended rearward from a rear end of the needle into the fluid cartridge for forming a fluid path from the fluid cartridge to the needle via the metal tube; and
an elongated syringe barrel including a lengthwise slot on its surface with the projection movably disposed therein, a locking trough proximate a curved rear end of the slot, and a flexible latch provided between the rear end of the slot and the locking trough;
whereby in an anesthetic dispensing operation pushing the arm forward will project the needle from the syringe barrel and move the projection to a forward end of the slot, and after the dispensing operation pulling the plunger rearward will retract the needle assembly into the syringe barrel until the projection moves from the forward end of the slot to the locking trough by passing the rear end of the slot and the latch for locking the projection in the locking trough.

2. The retractable syringe of claim 1, wherein the projection has a section of triangle and has its top surface inclined from the side facing the nose to a rear vertex.

3. The retractable syringe of claim 1, wherein the plunger further comprises a separate rubber ring disposed in close proximity to a rear of the disk.

4. The retractable syringe of claim 1, wherein each of the plunger, the arm, and the syringe barrel is formed of a plastic material.

5. The retractable syringe of claim 1, wherein a forming orientation of the flexible latch is toward the rear end of the slot.

6. The retractable syringe of claim 1, wherein the flexible latch will be deformed when the projection moves from the forward end of the slot to the locking trough and will not be deformed when the projection moves from the locking trough to the forward end.
